(19) United States
(12) Patent Application Publication
(10) Pub. No.: US 2017/0266030 A1
(43) Pub. Date: Sep. 21, 2017

(54) BANDAGE FOR THE BACK OR PELVIS

(71) Applicant: BAUERFEIND AG, Zeulenroda-Triebes (DE)

(72) Inventor: Heinrich HESS, Kleinblittersdorf (DE)

(73) Assignee: BAUERFEIND AG, Zeulenroda-Triebes (DE)

(21) Appl. No.: 15/505,356

(22) PCT Filed: Aug. 21, 2015
(86) PCT No.: PCT/EP2015/069272

§ 371 Date: Feb. 21, 2017

(30) Foreign Application Priority Data
Aug. 22, 2014 (DE) 10 2014 012 654.4

(51) Int. Cl. A61F 5/32; A61H 39/04; A61H 7/00; A61H 11/00; A61F 5/02; A61H 1/00

(52) U.S. Cl. CPC A61F 5/32; A61F 5/028; A61H 1/008; A61H 7/002; A61H 11/00; A61H 39/04; A61H 2201/1626; A61H 2201/163; A61H 2201/1692

(57) ABSTRACT

A bandage for the back or pelvis has adjacent pads mounted on an inner face of a supporting piece in a back area of the bandage, respectively one fastener strap piece for applying the bandage by joining together the ends of the two fastener strap pieces attaching at the opposite edge regions of the supporting piece, and two tensioning belts being assigned to the supporting piece, which are guided in such a manner that the pads approach each other when tensioning the tensioning belts and apply a pressure dependent on a tension force acting on the tensioning belts onto the relevant body part.

The present invention relates to a bandage for the back or pelvis having two adjacent pads, which are mounted on the inner face of a supporting piece in the back area of the bandage, respectively one fastener strap piece attaching at the opposite edge regions of the supporting piece for applying the bandage by joining together the ends of the two fastener strap pieces, and two tensioning belts being assigned to the supporting piece, which are guided in such a manner that the pads approach each other when tensioning the tensioning belts and apply a pressure dependent on a tension force acting on the tensioning belts onto the relevant body part.

Such a bandage is disclosed in DE 10 2011 000 953 A1. In this case, two tensioning belts are provided, which are attached on the outer face of the supporting piece. Guiding the belt to the abdominal side of the bandage in this instance occurs either in a crossed manner or via a deflection piece. The first embodiment has the disadvantage that the person putting on the bandage acts, when tightening the tensioning belts, upon the left pad using the right tensioning belt and upon the right pad using the left tensioning belt so that the bandage easily may be tightened in the wrong manner, which may be disadvantageous in particular for single-sided pain conditions, for example, pain conditions on only the right side or only on the left side. In the second embodiment, the deflected tensioning belts rub against each other during tightening.

The technical problem of the present invention is to provide a bandage for the back or pelvis which overcomes the disadvantages from the prior art.

Furthermore, a bandage for the back or pelvis is to be provided, for which the tensioning belts enable a more direct application of force onto the pads.

Also, a bandage for the back or pelvis is to be provided, which enables a flat and compact structure of the tensioning belt system.

The present invention solves the underlying technical problem by the subject of Claim 1.

The present invention solves the underlying technical problem, in particular, by a bandage for the back or pelvis having adjacent pads, which are mounted on the inner face of a supporting piece in the back area of the bandage, respectively one fastener strap piece attaching at the opposite edge regions of the supporting piece for applying the bandage by joining together the ends of the two fastener strap pieces, two tensioning belts being assigned to the supporting piece, which are guided in such a manner that the pads approach each other when tensioning the tensioning belts and apply a pressure dependent on a tension force acting on the tensioning belts onto the relevant body part, and the tensioning belts being connected to the supporting piece on the inner face of the supporting piece in the area of the pads.

It is shown that a connection of the tensioning belts on the inner face of the supporting piece in the area of the pads there attached advantageously enables a direct application of force from the tensioning belts onto the area of the pads and onto the pads themselves. In doing so, the tensioning belts may directly terminate in and act at the pads. This technical solution achieves that pain conditions in only one sacroiliac joint may also be advantageously influenced.

In a preferred embodiment, respectively one tensioning belt runs on the outer face of respectively at least one portion of a fastener strap piece.

Preferably, the supporting piece has at least one opening through which the tensioning belts may be guided to the inner face of the supporting piece.

Preferably, the supporting piece has two openings, and a tensioning belt is guided through respectively one opening to the inner face of the supporting piece.

In a preferred embodiment, the tensioning belts run partially on the outer face of the supporting piece, and the supporting piece has at least one opening through which the tensioning belts are guided to the inner face of the supporting piece.

The at least one opening, in particular the two openings, enable(s) in an advantageous manner that the tensioning belts may be guided from the inner face to the outer face of the supporting piece and, thus, on the one hand, a connection of the tensioning belts according to the present invention is enabled on the inner face of the supporting piece in the area of the pads and, on the other hand, a conventional guiding of the tensioning belts and possible tension straps on the outer face, in particular in the front area of the bandage, is simultaneously enabled, so that the person wearing the bandage may grip and use the tensioning belts or possible tension straps without further action. Furthermore, the at least one opening, through which the tensioning belts are guided from the outer face to the inner face of the supporting piece, has the advantage that here a deflection may occur, which enables a simpler more easily gliding and symmetrical application of force according to the cable-pull principle. Despite the deflection, the tensioning belts also advantageously run barely in opposite directions and do not rub against each other because the opposite running section of one tensioning belt may run on the inner face of the bandage.

For this reason, the at least one opening is located in an advantageous manner at the preferred deflection pieces according to the present invention.

Preferably, the first tensioning belt is guided through a first opening in the supporting piece to the inner face of the supporting piece and the second tensioning belt is guided through a second opening in the supporting piece to the inner face of the supporting piece.

Preferably, the at least one opening is a slit in the supporting piece. Preferably, the two openings are each one slit in the supporting piece, in particularly preferably having a border. This serves in an advantageous manner as a reinforcement so that the slits are not able to tear.

Preferably, the tensioning belts are each guided via a deflection piece mounted at the supporting piece by reversing the direction from the area of the pads to the respective edge region of the supporting piece.

Preferably, the deflection pieces are attached on the outer face of the supporting piece.

Preferably, a deflection lug attached in the center of the supporting piece forms the two deflection pieces.

Preferably, it is provided that the first tensioning belt is guided through a first slit from the inner face of the bandage to the outer face and the second tensioning belt is guided through a second slit from the inner face of the bandage to the outer face of the bandage and, then, the tensioning belts are deflected via the deflection lug attached to the center of the supporting piece and are guided on the outer face of the bandage to the front side of the bandage.

This enables a particularly flatly designed embodiment in the back area of the bandage.

In a preferred embodiment, the pads are reversibly attachable at the supporting piece, particularly preferably by a Velcro® connection. This has the advantage that the pads may be individually positioned and, in doing so, the force application acting through the tensioning belts may be also individualized. Preferably, the pads are mountable in that area of the supporting piece, where the tensioning belts are connected to the supporting piece. Preferably, the connection elements for the pads are, thus, in the area where the tensioning belts are connected to the supporting piece.

Suitable pads are known to the skilled person. Preferably, the pads have nubs. When the pads are moved, these nubs act in terms of a friction massage on the “trigger points” influencing the pain in the soft tissue, for example, in the muscle. In one embodiment, it may be provided that the pads may be supplied with heating elements or cooling elements. A preferably reversible attachability of the pads enables to easily exchange or replace such heating elements or cooling elements.

To be able to mount different pads at the bandage for the back or pelvis, in a preferred embodiment, the supporting piece is provided by a Velcro® fastener surface matching the respective Velcro® fastener surface of the pads, so that different pads may be exchanged in an easy manner via the Velcro® fasteners.

The tensioning belts are preferably connected to the supporting piece in the region of the pads. In so doing, it may be advantageously provided that the tensioning belts are connected to the supporting piece in the region of the pads, in particular, in the same location where the pads are connected to the supporting piece, which enables a particularly good application of force onto the pads.

Preferably, respectively one tensioning belt is connected at respectively one fastening area of a pad to the supporting piece, in particular, is directly there connected to the supporting piece.

In an alternative embodiment, the tensioning belts are connected to the supporting piece via respectively one banner-like fastening element located at the supporting piece. Such a banner-like fastening element, also know as a flap, enables a particularly good application of force of the tension forces of a tensioning belt directly onto the area of a pad.

The tension of the tensioning belts may be advantageously caused in that each tensioning belt respectively leads to and terminates in a lug element in the form of a tensioning lug through which respectively one tension strap is guided, which is guided by its one end to a fastener tension piece and, by its other end free to be gripped in such a manner that, when tensioning the two tension straps, the supporting piece is contracted and the pads approach each other. In this manner, the free ends of the two tension straps are able to be manually pulled, for the reason of which the tension straps via the deflection lugs exert a tension which is continued to the tensioning belts and, for this reason, exerts a tension running in the opposite direction onto the supporting piece, through which the supporting piece is contracted and the pads approach each other.

Preferably, the supporting piece and the areas of the fastener strap pieces adjacent to the supporting piece have the same elasticity.

Preferably, the supporting piece and at least the halves of the fastener strap pieces adjacent to the supporting piece have the same elasticity. Preferably, the supporting piece and the halves of the fastener strap pieces adjacent to the supporting piece have the same elasticity.

Surprisingly, it is shown that for the embodiment according to the present invention of the tensioning belts it is not necessary that the supporting piece has to be more flexible than the adjacent fastener strap pieces, such as it so far has been realized, for example, by the the SacroLoc® Orthese of Bauerfeind AG, Zeulenroda-Triebes, Germany, based on DE 10 2011 000 953 A1. For such bandages for the back or pelvis from the prior art, the supporting piece is more flexibly designed than the adjacent fastener strap pieces, so that when tightening the tensioning belts, the tension force primarily acts in the region of the pads located at the supporting piece. In so doing, the supporting piece and the adjacent fastener strap pieces may, however, not be manufactured from the same material and as one piece. It is now shown that for a bandage for the back or pelvis according to the present invention, the supporting piece may be equally elastic as the adjacent areas of the fastener strap pieces and the force of the tensioning belts still acts as desired primarily in the area of the support belt and the pads. Thus, an embodiment, in which the supporting piece and the adjacent areas of the fastener strap pieces are manufactured from the same material and even as one piece, is advantageously possible.

In this instance, the supporting piece may be preferably delineated from the adjacent fastener strap pieces by respectively one cross-bracing.

In an advantageous manner, it is also shown that the supporting piece may be manufactured from a very light and air-permeable knitted fabric. For this reason, the supporting piece is preferably manufactured from a knitted fabric. Preferably, the supporting piece is manufactured from a very light and air-permeable knitted fabric.

Preferably, the supporting piece and the halves of the fastener strap pieces adjacent to the supporting piece are manufactured from a very light and air-permeable knitted fabric.

Preferably, the supporting piece has an area made from a knitted fabric which has an open structure, which is simultaneously stretchable in the longitudinal direction as well as also in the lateral direction.

Preferably, the supporting piece has an area made from a knitted fabric which has an open structure made from a plurality of partial elements, the open structure simultaneously being stretchable in the longitudinal direction as well as also in the lateral direction, and the partial elements of the structure respectively having a width of at least 2.0 mm and no more than 10.0 mm in the stretched state.

Preferably, the partial elements of the knitted fabric structure respectively have a width of at least 2.0 mm and no more than 10.0 mm in the stretched state. Preferably, the partial elements of the knitted fabric structure respectively have a height of at least 2.0 mm and no more than 10.0 mm in the stretched state.

Preferably, the supporting piece has an area of a knitted fabric which is simultaneously stretchable in the longitudinal direction as well as also in the lateral direction, the knitted fabric having holes which respectively have a width and/or a height of at least 2.0 mm and no more than 10.0 mm in the stretched state. Preferably, the knitted fabric has a net structure.

Surprisingly, it is shown that such a knitted fabric may be advantageously used as the primary component of the supporting piece, resulting in a particularly light and air-permeable supporting piece.

Advantageously, such a knitted fabric may be also used for the fastener strap pieces, in particular, for the portions of the fastener strap pieces adjacent to the supporting piece.

Knitted fabrics, also called knitwear, are fabrics which are industrially manufactured from yarn systems on a knitter by stitch formation. Knitted fabrics belong to knits. A distinction is made between weft knits and warp knits. While during knitting or crocheting one stitch next to the other is produced, thus, the yarn runs horizontally along a row of stitches, for knitwear, the yarn forms stitches situated one above the other and the yarn runs perpendicular and, together with the adjacent yarn, forms a knitted web. A warp knit is manufactured using many threads and at least as many needles. When warp knitting, the threads run vertically and are captured by the needles and are pulled through the previous row of stitches.

Warp knits are, for example, used in the form of nets, for example, in the form of luggage nets in cars. The nets may be designed in an elastic or non-elastic manner.

Preferably, the knitted fabric has an open structure which is simultaneously stretchable in the longitudinal direction as well as also in the lateral direction. Preferably, the partial elements of the open structure respectively have a width of at least 2.0 mm up to no more than 10.0 mm in the stretched state. Preferably, the partial elements of the open structure have, alternatively or additionally, respectively a height of at least 2.0 mm up to no more than 10.0 mm in the stretched state.

According to the present invention, the knitted fabric is preferably designed as warp knits. According to the present invention, the knitted fabric is preferably designed as a net. Thus, the knitted fabric is preferably netted, that is, is an open-pored planar material having regular stitches formed as openings. The openings may be, for example, formed in a rhombical, square, hexagonal, diamond-shaped or a honeycomb manner. Preferably, the net structure is made of a plurality of equal or at least similar partial structures. Within the context of the present invention, a partial structure is understood as a stitch or an opening, in particular a hole, and threads surrounding and bounding the stitch or opening.

Within the context of the present invention, an open structure of a knitted fabric is in particular understood as a net structure, which has a plurality of holes and stitches, in particular bigger holes, preferably holes having a width and/or height of at least 2.0 mm, more preferably of at least 3.0 mm in the stretched state. Thus, an open structure is in particular an open-pored net.

The skilled person may conclude embodiment options of such a preferred knitted fabric from DE 10 2012 017 722 A1.

In the present application text, all data regarding height and/or width of the holes, partial elements and/or stitches relate to the height and/or width of the holes, partial elements and/or stitches in the stretched state of the knitted fabric in the bandage, as long as not indicated otherwise.

Within the context of the present invention, the term “in the stretched state” is understood as that state of the knitted fabric which is present when wearing the bandage. According to the present invention, the knitted fabric is preferably not used in the non-stretched state, but preferably in a slightly pre-stretched state. The knitted fabric is preferably not stretched to a maximum in the bandage but may preferably be further stretched when applying the bandage to the body of a human.

In a preferred embodiment of the present invention, the knitted fabric is a warp knit. In a preferred embodiment, the knitted fabric has no pile yarn.

In a preferred embodiment, the knitted fabric, which is in particular a warp knit, is characterized by the fact that the knitted fabric has no pile yarn and an open structure, which is simultaneously stretchable in the longitudinal direction as well as also in the lateral direction.

In a preferred embodiment, the knitted fabric has a ground thread and at least a further thread, in particular a weft thread. In a preferred embodiment, the thickness of the ground thread is greater than the thickness of the at least one additional thread, in particular of the weft thread.

For bandages for the back or pelvis from the prior art, the supporting piece and the adjacent fastener strap pieces are designed as a knit. In comparison, the knitted fabric may not only be designed in such a manner that it is stretchable, in particular simultaneously stretchable, in the longitudinal and the lateral direction, but also that the knitted fabric is designed so that it corresponds with high precision to required force expansion specifications, thus, that the knitted fabric when applying a specific force is able to feature a desired and specific stretch. Moreover, the knitted fabric is, owing to the holes, breathable, preferably having a width and/or a height of at least 2 mm and no more than 10 mm in the stretched state. It is shown that the knitted fabric has a significantly better breathability than knits so far used in the prior art. In so doing, a heat build-up is prevented so that perspiration is reduced. The knitted fabric has an elasticity which results in a good adaptation to the body shape, the two-dimensional plane adapting in an optimal manner to the three-dimensional body of the patient. This may be achieved in particular by forming the openings in the shape of diamonds.

In an advantageous manner, the knitted fabric may also generate new design effects, in particular structural effects, because longitudinal strips in different yarn colors are possible.

Advantageously, the knitted fabric is stretchable in the longitudinal as well as also in lateral direction.

In a preferred embodiment, the open structure of the knitted fabric is a diamond or honeycomb structure.

In a preferred embodiment, the partial elements of the structure, in particular of the diamond structure or honeycomb structure in the stretched state, preferably have, when stretched to a maximum, respectively a width and/or height of at least 2 mm, preferably at least 2 and no more than 10 mm, preferably at least 3 and no more than 8 mm, preferably at least 3 and no more than 7 mm, preferably at least 3 and no more than 6 mm, preferably at least 5 and no more than 6 mm.

In a preferred embodiment, the partial elements have an equal extension in the longitudinal and in the lateral direction. In the stretched state, the height and the width of the partial elements of the structure present in the knits are preferably approximately equal in size, preferably are equal in size.

In a preferred embodiment, the knitted fabric is made of a thread, preferably a warp thread having a yarn count or thickness of at least 100 dtex, more preferably of at least 250 dtex. In a preferred embodiment, the knitted fabric is made of a thread, preferably a warp thread having a yarn count or thickness of at least 500 dtex. In a preferred embodiment, the knitted fabric is made of a thread, preferably a warp thread having a yarn count or thickness of at least 700 dtex. In a preferred embodiment, the knitted fabric is made of a thread, preferably a warp thread having a yarn count or thickness of no more than 10.5 ktex. In a preferred embodiment, the knitted fabric is made of a thread, preferably a warp thread having a yarn count or thickness of at least 900 dtex up to no more than 999 dtex.

In a preferred embodiment, the knitted fabric expands by approximately 30% at an applied force of 1.0 through 5.0 N/cm, preferably approximately 2.5 N/cm. In a particularly preferred embodiment, the knitted fabric expands approximately 30% at an applied force of 1.0 through 5.0 N/cm, preferably 2.5 N/cm.

Preferred embodiments also result from the dependent claims.

The present invention is described in greater detail on the basis of the subsequent figures and examples, but the shown examples are not to be understood in a restrictive manner.

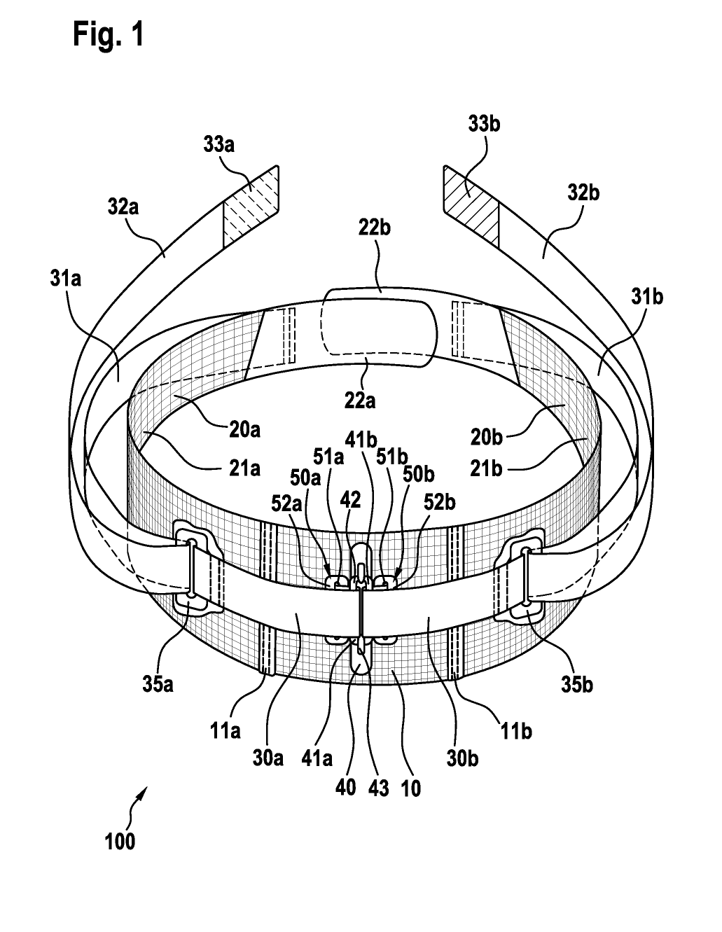

FIG. 1 shows a bandage for the back or pelvis according to the present invention with a view toward the outer face of the back area and of the supporting piece;

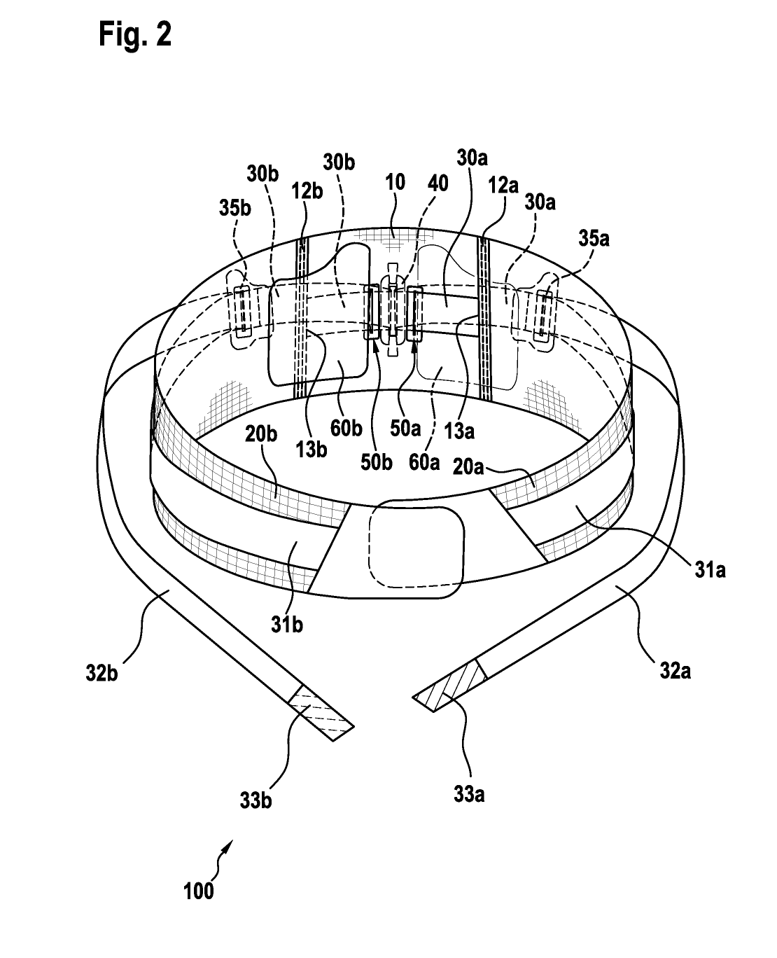

FIG. 2 shows the bandage from FIG. 1 with a view toward the inner face of the back area and of the supporting piece;

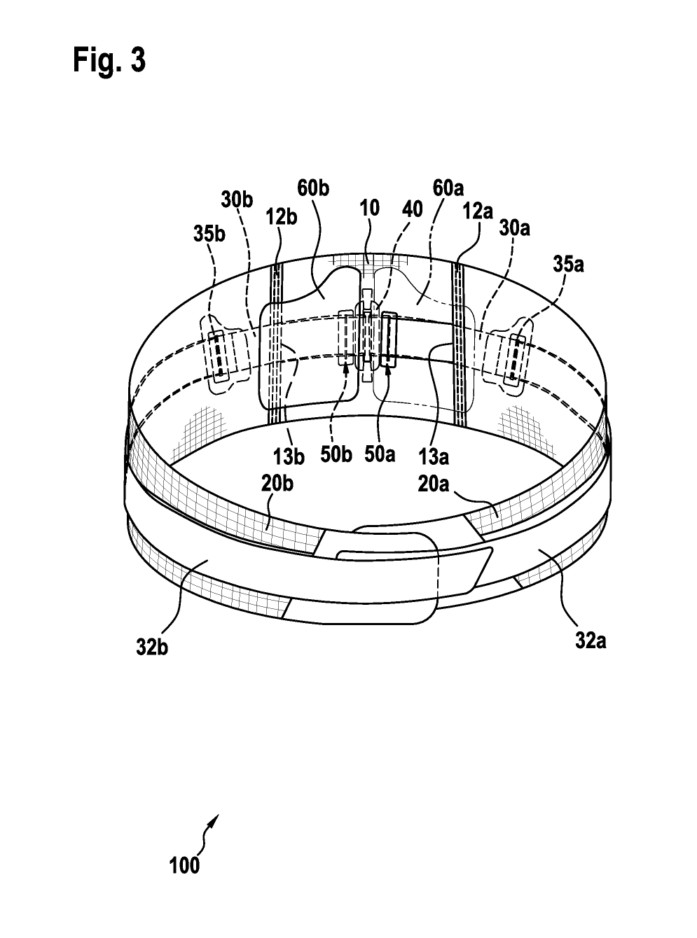

FIG. 3 shows the bandage from FIG. 2 having tightened tensioning belts and pads drawn near to each other;

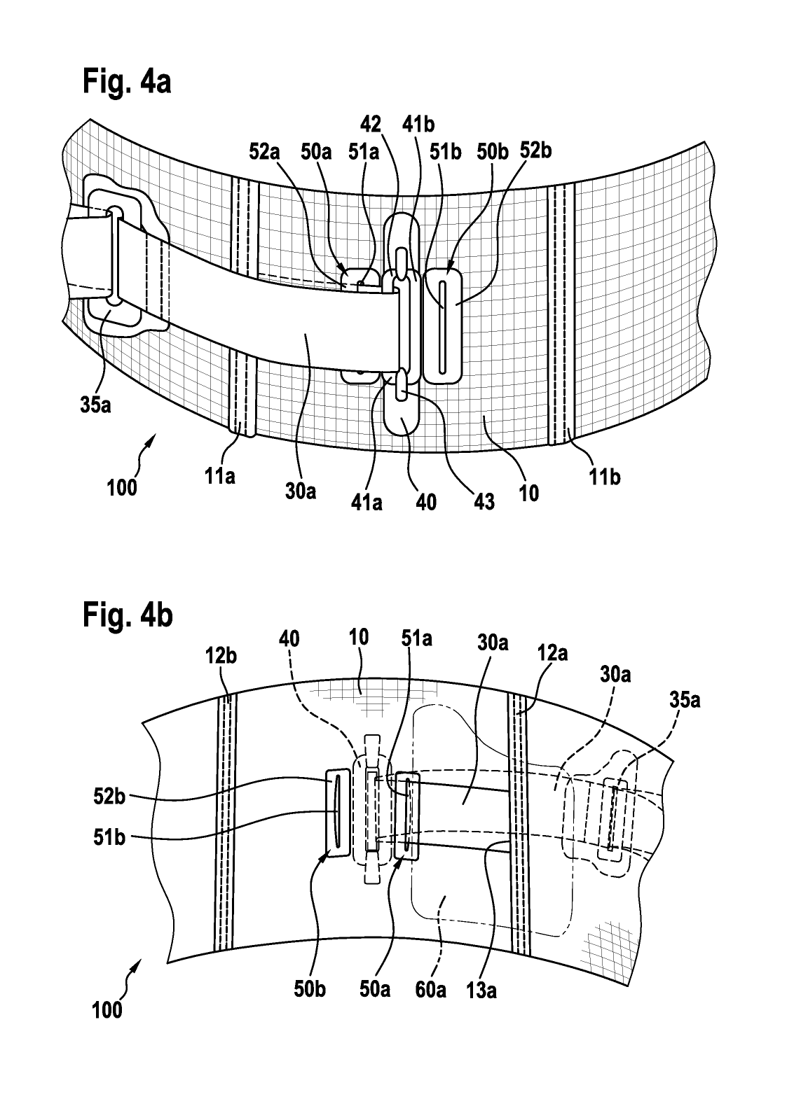

FIG. 4 shows a detail of the back area having the supporting piece of the bandage with a view toward the outer face of the bandage (FIG. 4a) and toward the inner face of the bandage (FIG. 4b);

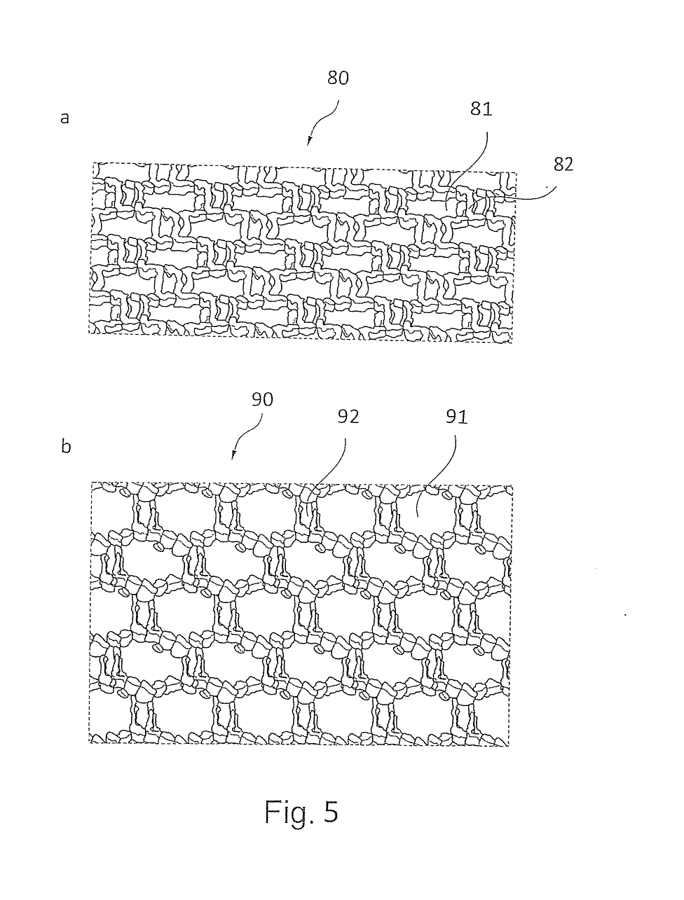

FIG. 5 shows a detail of the warp knit preferably used for the supporting piece and the adjacent partial areas of the fastener strap pieces, having a plurality of partial elements. In FIG. 5a, the warp knit is shown in a non-stretched state. In FIG. 5b, the warp knit is shown in a stretched state.

### EXAMPLES

### Example 1

### A Preferred Embodiment of the Bandage for the Back or Pelvis According to the Present Invention

FIG. 1 shows a bandage (100) according to the present invention with a view on the outer face of the elastic supporting piece (10). The two fastener strap pieces (20a, 20b) connect at the sides of the supporting piece (10). The supporting piece (10) as well as also the areas (21a, 21b) of the fastener strap pieces (20a, 20b) adjacent to the supporting piece may be advantageously manufactured from the same material and even as one piece, for example, from a stretchable knitted fabric, as it is shown in FIG. 5. Even when the support piece (10) and the adjacent areas (21a, 21b) of the fastener strap pieces (20a, 20b) are manufactured as one piece, they may still be delimited from each other by mounted reinforcements in the form of cross-bracings (11a, 11b). The fastener strap pieces (20a, 20b) may be joined with each other in their end region (22a, 22b), for example, via a respective Velcro® connection. After applying the bandage and closing of the ends (22a, 22b) of the fastener strap pieces (20a, 20b), the bandage (100) may be further tightened via the tensioning belts (30a, 30b). In so doing, the ends of the tensioning belts are connected to the supporting piece on the inner face of the supporting piece (10) facing away from the viewer in FIG. 1. The tensioning belts (30a, 30b) then run on the inner face to the center area of the supporting piece (10) and are there guided through openings (50a, 50b) to the outer face of the supporting piece (10). The openings (50a, 50b) are formed as slits (51a, 51b), which are surrounded by a reinforcement (52a, 52b), so that a tearing of the supporting piece material by the tension forces of the tensioning belts (30a, 30b) is prevented. On the outer face of the supporting piece (10), the tensioning belts (30a, 30b) are then deflected via deflection pieces (41a, 41b), so that they run in the direction of the respective fastener strap pieces (20a, 20b). The deflection pieces (41a, 41b) are advantageously formed by a deflection element (40), including a deflection lug (42) which is connected to the supporting piece (10) via a lug attachment element (43). In the area of the fastener strap pieces (20a, 20b), the tensioning belts (30a, 30b) end at the lug elements in the form of tensioning lugs (35a, 35b), through which respectively one tension strap (31a, 31b, 32a, 32b) is pulled. Preferably, the lug elements (35a, 35b) are not directly attached at the fastener strap pieces (20a, 20b). A tension in the manner of a pulley is applied onto the tensioning belts (30a, 30b) by the tension straps (32a, 32b) when tightened so that the tensioning belts (30a, 30b) apply a tension force on the inner face of the supporting piece (10), and the supporting piece contracts or, when loosening the tensioning belts (31a, 31b, 32a, 32b), relaxes. The tension straps (32a, 32b) may be connected to each other by their ends (33a, 33b), for example, via a Velcro® fastener. Alternatively, an embodiment may of course also be provided, in which the tensioning belts (30a, 30b) are guided completely to the front and there may be directly tightened and be connected to each other.

FIG. 2 shows the bandage (100) according to FIG. 1 in a position with a view toward the inner face of the elastic supporting piece (10) having the pads (60a, 60b) thereto connected. In this instance, the right pad (60a) in the direction of viewing is only indicated to better see the tensioning belt (30a) situated behind. The supporting piece (10) is delimited from the adjacent fastener strap pieces (20a, 20b) by a reinforcement (12a, 12b) which, however, is a component of the supporting piece (10). In this instance, the reinforcements advantageously serve as Velcro® connection elements for the pads (60a, 60b), so that there the pads (60a, 60b) may be attached to the supporting piece (10) with the aid of a Velcro® connection. In these pad-fastening areas (12a, 12b), the tensioning belts (30a, 30b) are also respectively attached to the supporting piece (10) in attachment areas (13a, 13b). The tensioning belts (30a, 30b) are then guided on the inner face of the supporting piece (10) to the openings (50a, 50b) and from there to the outer face of the supporting piece (10). There, the tensioning belts are deflected by the deflection element (40) and are guided to the lug elements in the form of tensioning lugs (35a, 35b), where the tensioning belts, as it has already been described for FIG. 1, are connected to the tension straps (31a, 31b, 32a, 32b), and the tension straps at their ends (33a, 33b) are closeable via a Velcro® connection.

FIG. 3 shows the bandage (100) from FIG. 2 having the supporting piece (10) and the fastener strap pieces (20a, 20b) in the closed state. In this instance, the tension straps (32a, 32b) are also tightened and connected to each other. Thus, they apply a tension force onto the tensioning belts (30a, 30b) via the lug elements (35a, 35b). Owing to the deflection of the tensioning belts (30a, 30b) via the deflection element (40) and the subsequent guiding of the tensioning belts (30a, 30b) through the openings (50a, 50b) to the inner face of the supporting piece (10), this tension force directly acts on the attachment areas (13a, 13b) and the pads (60a (here only indicated), 60b) mounted to the attachment areas at fastening areas (12a, 12b), for example via Velcro® connections, so that the pads are more strongly contracted toward the center of the supporting piece (10). For this tensile stress of the two tensioning belts (30a, 30b), it thus results a contraction of the supporting piece (10) on the basis of the inversion of the direction via the deflection element (40) and, for this reason, a respective approach of the pads (60a, 60b) mounted thereto. In this instance, the tension force of the tensioning belts (30a, 30b) advantageously acts directly onto the inner face of the supporting piece (10) and, for this purpose, onto that area (12a, 12b) of the supporting piece (10) where the pads (60a, 60b) are attached.

FIG. 4 shows an enlarged detail of the bandage (100) in the area of the supporting piece (10). For this purpose, the bandage (100) in FIG. 4a may be seen from the outside and in FIG. 4b from the inside. For a better illustration, only one tensioning belt (30a) having an assigned lug element (35a) is shown for this purpose. The course of the second tensioning belt here not shown results from FIGS. 1 through 3.

FIG. 4a again shows the reinforcement elements (11a, 11b), which delineate the supporting piece (10) from the adjacent fastener strap pieces. The deflection element (40) again includes a deflection lug (42), which forms the two deflection pieces (41a, 41b) and is attached to the supporting piece (10) via a lug attachment element (43). The openings (50a, 50b), through which the tensioning belts (30a shown) are guided to the inner face of the supporting piece (10), are formed by slits (51a, 51b) surrounded by a reinforcement (52a, 52b).

In FIG. 4b, the deflection element (40) is only indicated because it is located on the outer face of the supporting piece (10). The tensioning belts (30a here shown) are guided through the openings (50a, 50b) formed as slits (51a, 51b) provided with a border (52b) to the inner face, where they are connected to the supporting piece (10) via attachment areas (13a). The fastening areas (12a, 12b) of the pads (60a here indicated), which for example may be attached to the supporting piece (10) via Velcro® connections, are also located in these attachment areas.

### Example 2

### Warp knit for the Supporting Piece and the Adjacent Partial Areas of the Fastener Strap Pieces

A warp knit has been machine-made from a double-covered raw rubber yarn. The stitch-forming yarn used a titer of 940 dtex. FIG. 5 shows a detail of this warp knit (80, 90). The warp knit (80, 90) is netted. The warp knit (80, 90) is made of a plurality of partial elements, each partial element being made of an opening (81, 91) and the yarn (82, 92) surrounding the opening (81, 91), which simultaneously also delineates the opening (81, 91) and, thus, specifies the size of the opening. In FIG. 5a, the warp knit (80) is not stretched. In FIG. 5b, the warp knit (90) is stretched by a specified force. By stretching the warp knit (90), the structure (92) opens, so that the openings (91) increase in size in the direction of stretching. Such a warp knit improves the wearing comfort of the bandage for the back or pelvis according to the present invention by the reduced weight and the air-permeable openings (91).

1-10. (canceled)

11. A bandage for the back or pelvis comprising:
a support piece in a back area of the bandage;
adjacent pads mounted on an inner face of the supporting piece;
first and second fastener strap pieces attached at first and second opposite edge regions of the supporting piece, respectively, for applying the bandage by joining together ends of first and second fastener strap pieces;
first and second tensioning belts assigned to the supporting piece which are guided such that the adjacent pads approach each other when tensioning the tensioning belts for applying a pressure dependent on a tension force acting on the tensioning belts onto the relevant body part, the tensioning belts connected to the supporting piece on the inner face of the supporting piece in an area of the pads.

12. The bandage for the back or pelvis of claim 11, wherein the tensioning belts run partially on an outer face of the supporting piece and the supporting piece has at least one opening through which the tensioning belts are guided to the inner face of the supporting piece.

13. The bandage for the back or pelvis of claim 11, wherein the first tensioning belt is guided through a first opening in the supporting piece to the inner face of the supporting piece and the second tensioning belt is guided through a second opening in the supporting piece to the inner face of the supporting piece.

14. The bandage for the back or pelvis of claim 11, wherein the at least one opening is a slit in the supporting piece.

15. The bandage for the back or pelvis of claim 11, wherein the tensioning belts are each guided via a deflection piece mounted at the supporting piece by reversing the direction from the area of the pads to the respective edge region of the supporting piece.

16. The bandage for the back or pelvis of claim 15, wherein the deflection piece is attached on the outer face of the supporting piece.

17. The bandage for the back or pelvis of claim 15, further comprising a deflection lug attached in a center of the supporting piece which forms the two deflection pieces.

18. The bandage for the back or pelvis of claim 11, wherein respectively one tensioning belt is connected in respectively one attachment area of a pad to the supporting piece.

19. The bandage for the back or pelvis of claim 11, wherein the supporting piece has an area made from a knitted fabric which has an open structure made from a plurality of partial elements, wherein the open structure simultaneously is stretchable in the longitudinal direction as well as also in the lateral direction, wherein the partial elements of the structure respectively have a width of at least 2.0 mm and no more than 10.0 mm in the stretched state.

20. The bandage for the back or pelvis of claim 11, wherein the supporting piece and a half of the fastener strap pieces adjacent to the supporting piece have equal elasticity.
